(19) United States
(12) Patent Application Publication
(10) Pub. No.: US 2009/0112114 A1
(43) Pub. Date: Apr. 30, 2009

(54) Method and system for self-monitoring of environment-related respiratory ailments

(76) Inventors: Deepak V. Ayyagari, Vancouver, WA (US); Wai-Chung Chan, Redmond, WA (US)

Correspondence Address: SHARP LABORATORIES OF AMERICA, INC., 1320 PEARL ST., SUITE 228, BOULDER, CO, 80302, US

(21) Appl. No.: 11/999,569

(22) Filed: Dec. 6, 2007

Related U.S. Application Data
(60) Provisional application No. 61/000,507, filed on Oct. 26, 2007.

(51) Int. Cl. A61B 5/08

(52) U.S. Cl. 600/529

(57) ABSTRACT

Methods and systems for continual self-monitoring of respiratory health and components for use therewith. The present methods and systems and their related components improve the standard of core in respiratory health self-monitoring by providing continual and unobtrusive monitoring that accounts for environmental, physiological and patient background information, and is capable of yielding a complex array of respiratory health-preserving responses. In some embodiments, the present methods and systems leverage ubiquitous handheld electronic devices [e.g. cell phones and personal data assistants (PDA)] for respiratory health self-monitoring.

### CROSS-REFERENCE TO RELATED APPLICATION

This application claims priority benefits under 35 U.S.C. 119(e) from U.S. Provisional Patent Application No. 61/000,507 filed on Oct. 26, 2007.

### BACKGROUND OF THE INVENTION

The present invention relates to monitoring respiratory health outside of a clinical setting and, more particularly, to methods and systems for self-monitoring of environment-related respiratory ailments, such as asthma and rhinitis.

Asthma is a chronic disease in which breathing becomes constricted. Asthma can significantly impair human well-being and in the most severe cases can be life-threatening. Asthma sufferers often experience attacks outside of a clinical setting that are triggered by environmental conditions, such as a dust, temperature and humidity. Self-monitoring systems have been developed to assist asthma sufferers in monitoring their respiratory health outside of a clinical setting to manage the disease and prevent the onset and reduce the severity of attacks.

A self-monitoring system for asthma sufferers that reflects the current standard of care is the peak flow meter with generic health self-monitoring program. In this system, the patient blows air into a peak flow meter and the meter outputs data such as the rate of expiratory flow. The patient then either manually inputs the data from the meter into a computer or the data are automatically uploaded to a computer. A generic respiratory health self-monitoring program running on the computer applies the data and outputs to the patient a discrete respiratory health level determined using the data. For example, the program may output one of green, indicating no action is required; yellow, indicating medication should be taken; or red, indicating that the patient should visit a clinician.

Unfortunately, the above-described self-monitoring system is inadequate in several respects. First, the system is strictly episodic. The patient is only informed a health level when he or she blows into the peak flow meter and the data are input, which may happen only a few times a day. Second, the system is obtrusive. The patient must apply the meter to his or her mouth and blow into it in order to generate the data. Moreover, the patient in some cases must manually input the data into a computer, which is time-consuming and requires computer access. Third, the system makes the respiratory health determination based on limited data. The data provided by a peak flow meter do not provide a comprehensive assessment of lung function and do not provide any information about environmental conditions that may trigger an attack. Moreover, the generic health self-monitoring program does not consider patient background data that may be relevant to the health determination, such as behavior patterns, co-morbidities, medications, age, height, weight, gender, race and genetic background. Finally, the discrete output levels yielded by the system may not provide sufficiently detailed information.

### SUMMARY OF THE INVENTION

The present invention, in a basic feature, provides methods and systems for self-monitoring of respiratory health and components for use therewith. The present methods and systems and their related components improve the standard of care in respiratory health self-monitoring by providing regular and unobtrusive monitoring that accounts for environmental, physiological and patient background information, and is capable of yielding a complex array of respiratory health-preserving responses. In some embodiments, the present methods and systems leverage ubiquitous handheld electronic devices [e.g. cell phones and personal data assistants (PDA)] for respiratory health self-monitoring.

In one aspect of the invention, a method for respiratory health self-monitoring comprises the steps of receiving physiological data collected from a patient, receiving environmental data and generating respiratory health data for the patient based at least in part on the physiological data and the environmental data.

In some embodiments, the physiological data and the environmental data comprise data received on a mobile electronic device at regular intervals.

In some embodiments, the physiological data further comprise data received on a mobile electronic device episodically.

In some embodiments, the respiratory health data are further generated based at least in part on statically configured patient background data, such as behavior pattern data, co-morbidity data, medication data, age data, height data, weight data, gender data, race data and/or genetic background data.

In some embodiments, the respiratory health data comprise present health data generated using current physiological data and environmental data.

In some embodiments, the respiratory health data comprise health trend data generated using historical physiological data and environmental data.

In some embodiments, the respiratory health data comprise health cross-correlation data generated using historical physiological data and environmental data.

In some embodiments, the method further comprises the step of outputting the respiratory health data on a user interface of a mobile electronic device.

In some embodiments, the method further comprises the step of outputting a respiratory health alert in response to the respiratory health data. In some embodiments, the alert is outputted on a user interface of a mobile electronic device. In some embodiments, the alert is outputted on a clinician computer and/or family member computer.

In some embodiments, the method further comprises the steps of controlling an environment control system in response to the respiratory health data, such as activation or deactivation of an air conditioning, heating, humidification or ventilation system.

In some embodiments, the method further comprises the step of generating a predictive model for the patient in response to the respiratory health data.

In some embodiments, the physiological data comprise lung sound data, blood oxygen saturation (SpO2) data and/or pulse rate data.

In some embodiments, the environmental data comprise airborne particulate data, temperature data and/or relative humidity data.

In another aspect of the invention, a handset comprises at least one network interface and a processor communicatively coupled with the network interface wherein the network interface is adopted to receive at regular intervals physiological data from at least one physiological monitor and environmental data from at least one environmental monitor and the processor is adapted to generate respiratory health data for a patient operatively coupled to the at least one physiological monitor based at least in part on the physiological data and the environmental data.

In some embodiments, the network interface receives the physiological data and the environmental data via wireless links.

In yet another aspect of the invention, a body area network (BAN) comprises at least one physiological monitor operatively coupled to a patient, at least one environmental monitor and a handset communicatively coupled with the physiological monitor and the environmental monitor, wherein the handset generates respiratory health data for the patient based at least in part on physiological data acquired by the handset at regular intervals from the physiological monitor and the environmental monitor.

These and other aspects of the invention will be better understood by reference to the following detailed description taken in conjunction with the drawings that are briefly described below. Of course, the invention is defined by the appended claims.

### BRIEF DESCRIPTION OF THE DRAWINGS

FIG. 1 shows a communication system operative to facilitate respiratory health self-monitoring in some embodiments of the invention.

FIG. 2 shows the BAN of FIG. 1 in more detail.

FIG. 3 shows the handset of FIG. 2 in more detail.

FIG. 4 shows functional elements of the handset of FIG. 2 operative to facilitate respiratory health self-monitoring in some embodiments of the invention.

FIG. 5 shows a method for respiratory health self-monitoring in some embodiments of the invention.

### DETAILED DESCRIPTION OF A PREFERRED EMBODIMENT

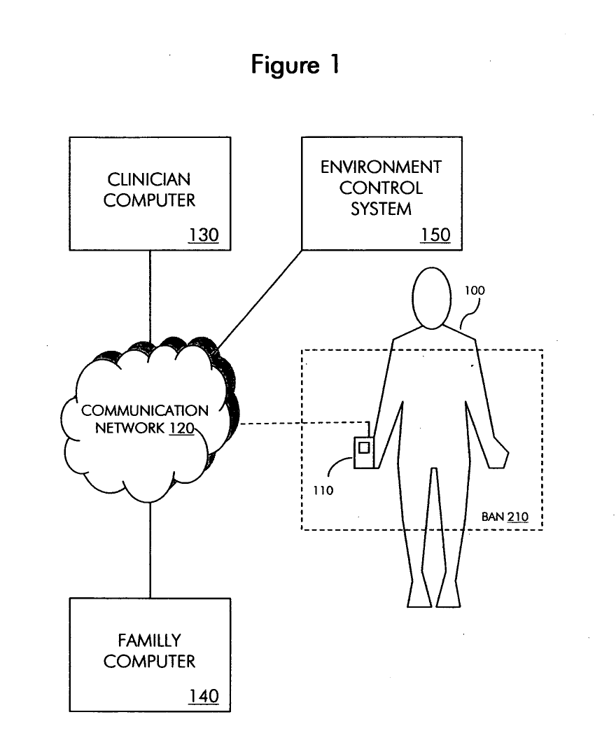

FIG. 1 shows a communication system operative to facilitate respiratory health self-monitoring in some embodiments of the invention. The system includes a handset 110 within a body area network (BAN) 210 in the immediate vicinity of a patient 100. Handset 110 is remotely coupled with a clinician computer 130 and a patient computer 140 via a communication network 120. Handset 110 is also communicatively coupled with an environment control system 150, either remotely via communication network 120 or locally via a separate wireless link.

Handset 110 is a handheld mobile electronic device operated by patient 100. Handset 110 may be a cellular phone, personal data assistant (PDA) or a handheld mobile electronic device that is dedicated to management of BAN 210, for example.

Clinician computer 130 is a computing device operated by a clinician who treats patient 100 or his or her agent. Clinician computer 130 may be a desktop computer, notebook computer, cellular phone or PDA, for example.

Family computer 140 is a computing device operated by a family member of patient 100. Family computer 140 may be a desktop computer, notebook computer, cellular phone or PDA, for example.

Environment control system 150 is a system adapted to regulate an indoor environment where patient 100 is located. Environment control system 150 may be an air conditioning, heating, humidification or ventilation system, for example.

Communication network 120 is a data communication network that may include one or more wired or wireless LANs, WANs, WiMax networks, USB networks, cellular networks and/or ad-hoc networks each of which may have one or more data communication nodes, such as switches, routers, bridges, hubs, access points or base stations, operative to communicatively couple handset 110 with clinician computer 130, family computer 140 and environment control system 150. In some embodiments, communication network 120 traverses the Internet.

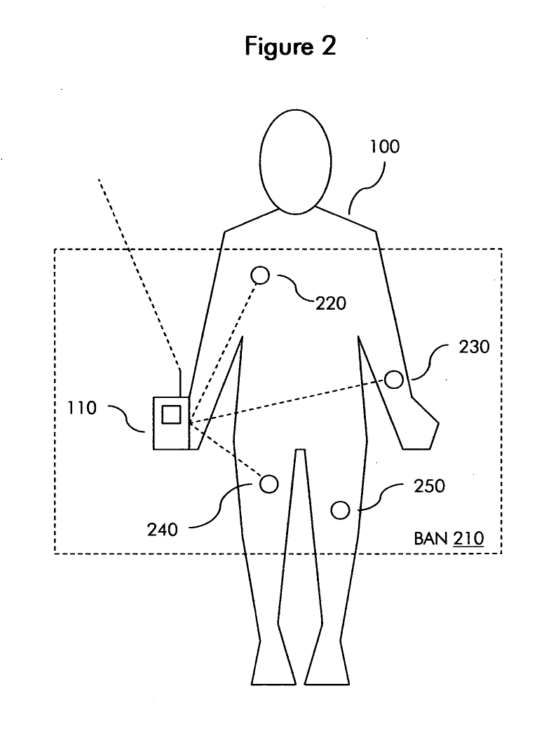

FIG. 2 shows BAN 210 in more detail. BAN 210 is a short-range network that operates in the immediate vicinity of patient 100. BAN 210 is illustrated as a fully wireless network, although in some embodiments BAN 210 may be fully or partly wired. BAN 210 includes a plurality of physiological monitors operatively coupled to patient 100, including at least one lung monitor 220 and at least one pulse monitor 230. BAN 210 also includes a plurality of environmental monitors, including at least one airborne particulate monitor 240 and at least one temperature/humidity monitor 250. Monitors 220, 230, 240, 250 are communicatively coupled with handset 110. Where connected by wireless segments, monitors 220, 230, 240, 250 and handset 110 communicate using a short-range wireless communication protocol, such as Bluetooth, Infrared Data Association (IrDa) or ZigBee. Where connected by wired segments monitors 220, 230, 240, 250 and handset 110 communicate using a short-range wired communication protocol, such as Universal Serial Bus (USB) or Recommended Standard 232 (RS-232). While environmental monitors 240, 250 are shown coupled to patient 100, in some embodiments one or more environmental monitors may be embedded in or attached to handset 110.

In some embodiments, lung monitoring is performed using phonospirometry or phonopneumography. In these embodiments, lung monitor 220 is a contact sensor or small microphone that captures the time domain waveform of lung sound. In some embodiments, lung sound is captured at a sampling frequency of at least 4000 Hz to permit detection of low frequency peaks indicative of wheezing. In other embodiments, lung monitoring may be performed using respiratory inductance plethysmography (RIP).

Pulse monitor 230 is a pulse oximeter that measures blood oxygen saturation (SpO2) level and pulse rate simultaneously. In some embodiments, pulse monitor 230 is placed on the wrist or finger of patient 100.

Airborne particulate monitor 240 is a sensor that measures particle density (e.g. in units of milligrams per cubic centimeter or number of particles per cubic meter). In some embodiments, particulate monitor 240 measures particle density for several ranges of particle sizes. In other embodiments, particulate monitor 240 measures overall particle density without regard to particle sizes. Particulate monitor 240 may generate an output voltage in proportion to particle density. For example, when there are few or no particles in the air, the output voltage may be approximately equal to a nominal voltage (e.g. one volt). When there are moderate airborne particle levels, the output voltage may meaningfully exceed the nominal voltage. When there are high airborne particle levels, the output voltage may approach a saturation voltage (e.g. three volts). Output voltage measurements may be taken at regular intervals, such as every 10 milliseconds.

Temperature/humidity monitor 250 measures ambient temperature and relative humidity. In some embodiments, a separate temperature monitor and humidity monitor may be deployed.

In some embodiments, other physiological and environmental monitors may be deployed to detect other representative or causative predictors of asthma attacks, for example, cockroach droppings, pesticides, cleaning agents, nitric oxide or heartbeat variation.

In some embodiments, a single monitor is used to acquire both physiological and environmental data. For example, a single monitor may capture environmental data and SpO2 level.

In some embodiments, a motion monitor is employed to determine the state of motion of patient 100, for example, whether patient 100 is moving, sifting, sleeping or standing. Such a motion monitor has an accelerometer for detecting acceleration and an associated algorithm for resolving the detected acceleration to a state of motion of patient 100. The accelerometer may be integral with a physiological or environmental monitor or may be a discrete unit. The associated algorithm may be integral with the motion monitor or handset 110.

Monitors 220, 230, 240, 250 have respective memories for temporarily storing their respective measured data.

Physiological data measured by lung monitor 220 and pulse monitor 230 and environmental data measured by dust monitor 240 and temperature/humidity monitor 250 are continually acquired by handset 110. In some embodiments, handset 110 acquires measured data by polling monitors 220, 230, 240, 250 at regular intervals and reading measured data from their respective memories. Monitors 220, 230, 240, 250 may be polled with the same frequency or with different frequencies. In some embodiments, handset 110 polls each monitor at least once per minute.

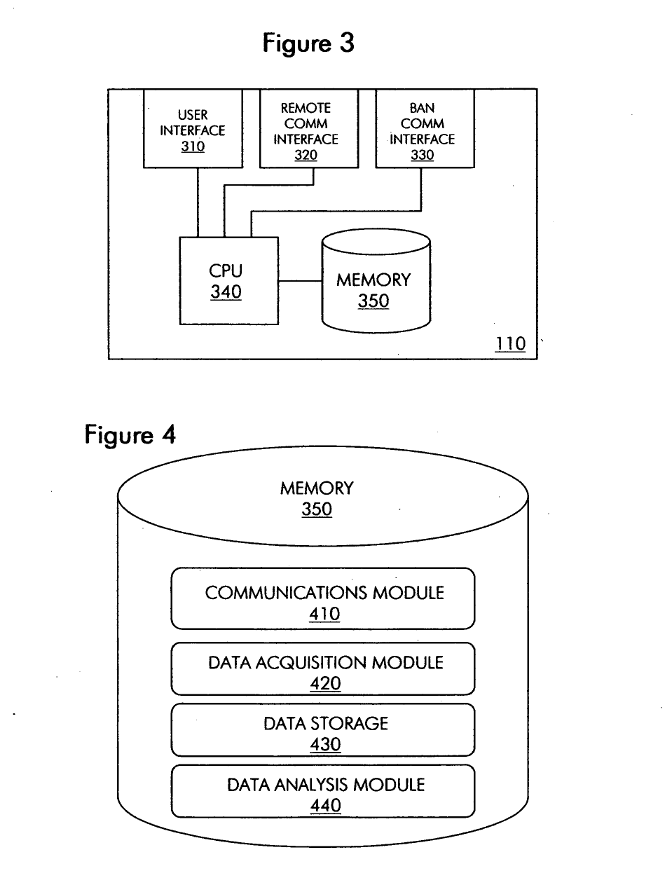

FIG. 3 shows handset 110 in more detail. Handset 110 includes a user interface 310 adapted to render outputs and receive inputs from patient 100. User interface 310 includes a display, such as a liquid crystal display (LCD) or light emitting diode (LED) display, and a loudspeaker for rendering outputs and a keypad and microphone for receiving inputs. Handset 110 further has a remote communication interface 320 adapted to transmit and receive data to and from communication network 120 in accordance with a wireless communication protocol, such as a cellular or wireless LAN protocol. Handset 110 further includes a BAN communication interface 330 adapted to transmit and received data to and from BAN 210. Handset 110 further includes a memory 350 adapted to store handset software, settings and data. In some embodiments, memory 350 includes one or more random access memories (RAM) and one or more read only memories (ROM). Handset 110 further has a processor 340 communicatively coupled between elements 310, 320, 330, 350. Processor 340 is adapted to execute handset software stored in memory 350, reference handset settings and data, and interoperate with elements 310, 320, 330, 350 to perform the various features and functions supported by handset 110.

FIG. 4 shows functional elements of handset 110 operative to facilitate respiratory health self-monitoring in some embodiments of the invention. The functional elements include a communications module 410, a data acquisition module 420 and a data analysis module 440. Modules 410, 420, 440 are software programs having instructions executable by processor 340 to acquire patient background data, physiological data and environmental data, store and retrieve such data to and from data storage 430, manipulate such data, generate respiratory health data for patient 100 and output alerts and environment control messages.

Communications module 410 supports remote communication interface 320 and BAN communication interface 330 in providing wireless communication protocol functions that enable handset 110 to transmit and receive data over communication network 120 and BAN 210, respectively. Wireless communication protocol functions include wireless link establishment, wireless link tear-down and packet formatting, for example. Where BAN 210 includes wired segments, communications module 410 also supports BAN communication interface 330 in providing wired communication protocol functions.

Data acquisition module 420 acquires patient background data, physiological data and environmental data and stores the acquired data in data storage 430. Patient background data is statically configured information that is input by patient 100 on user interface 310, or input by a clinician on clinician computer 130 and received on remote communication interface 320 via communication network 120. Patient background data is information specific to patient 100 that may render patient 100 more or less susceptible to environmental or physiological conditions that may cause or exacerbate respiratory ailment. Patient background data may include, for example, behavior patterns (e.g. exercise patterns, sleep patterns), co-morbidities [e.g. stress level, pulmonary hypertension, chronic obstructive pulmonary disease (COPD), bronchiectosis], medications, age, height, weight, gender, race, genetic background and general sense of well-being. Physiological and environmental data is information continually received on BAN communication interface 330 from monitors 220, 230, 240, 250. Data acquisition module 420 may poll monitors 220, 230, 240, 250 at a polling interval configured on handset 100 to continually acquire physiological and environmental data. Physiological data acquired from lung monitor 220 and pulse monitor 230 may include, for example, lung sound data, SpO2 data and pulse rate data. Environmental data acquired from airborne particulate monitor 240 and temperature/humidity monitor 250 may include, for example, particle density data, ambient temperature data and relative humidity data. In some embodiments, physiological and environmental data measurement and acquisition processes run continuously on monitors 220, 230, 240, 250 and data acquisition module 420 and measure/acquire physiological and environmental data with sufficient frequency to ensure that the current state of respiratory health of patient 100 is always known.

In some embodiments, data acquisition module 420 also acquires episodic physiological data on patient 100 through static configuration. For example, patient 100 may input on user interface 310 or a clinician may input on clinician computer 130 and transmit to handset 110 via communication network 120 at irregular intervals lung performance data obtained using a peak flow meter or spirometer (e.g. forced expiratory volume in one second).

Data analysis module 440 performs preprocessing functions that convert, where required, acquired physiological and environmental data into a form suitable for analysis. For example, data analysis module 440 separates lung sound from other noise (e.g. heartbeat, voice) in the time domain waveform of lung sound data acquired from lung monitor 220 and performs a Fast Fourier Transform (FFT) to convert the time domain waveform into a frequency domain representation so that the presence of low frequency peaks indicative of wheezing can be detected.

Data analysis module 440 applies patient background data, physiological data and environmental data to generate respiratory health data. Generated respiratory health data include present health data and health trend data. Present health data includes values for scientific parameters generated using physiological data and environmental data that are indicative of the current respiratory health of patient 100, such as current wheeze rate, crackle rate, pulse rate, respiratory rate, inspiratory duration, expiratory duration, SpO2 level, airborne particle levels, ambient temperature and relative humidity. Data analysis module 440 can determine the current respiratory rate, inspiratory duration and expiratory duration of patient 100 from the acquired time domain representation of lung sound and can determine the current wheeze and crackle rates of patient 100 from the derivative frequency domain representation of lung sound. Data analysis module 440 can determine overall airborne particle density from acquired output voltage measurements indicative of particle density and can also identify specific airborne irritants from such output voltage measurements. For example, if the output voltage pattern consists of several consecutive well above nominal output voltages it may indicate the presence of dense or thick irritants, such as cigarette smoke. If the output voltage pattern, on the other hand, consists of nominal output voltages interrupted by occasional output voltage spikes, it may indicate the presence of thin or less dense irritants, such as scattered pollen or dust. More generally, data analysis module 440 can determine one or more of presence, type, density, concentration or size of airborne particulates. Data analysis module 440 also generates patient-friendly present health data using scientific parameter values and patient background data. For example, data analysis module 440 may resolve patient background data and one or more of current wheeze rate, crackle rate, pulse rate, respiratory rate, inspiratory duration, expiratory duration, SpO2 level, airborne particle levels, ambient temperature and relative humidity to a respiratory health score between, for example, one and five. It will be appreciated that reducing present respiratory health to a simple numerical score for presentation to patient 100 may allow patient 100, who may lack medical expertise, to readily assess his or her present respiratory health. Data analysis module 440 adds present health data to a data history retained in data storage 430.

Generated respiratory health data include health trend data. Health trend data are indicative of a respiratory health trend experienced by patient 100. Data analysis module 440 determines a trend from historical data retained in data storage 430 for each scientific parameter. The trend may be as rudimentary as upward or downward or more complex, such as rapidly accelerating, slowly accelerating, stable slowly decelerating or rapidly decelerating.

In addition, data analysis module 440 may determine cross-correlations between different scientific parameters that suggest the possible onset of an asthma attack. For example, correlations may be detected between a certain concentration of allergen particles and the onset of wheezing by patient 100. These cross-correlations can be applied to generate a predictive model that is individually tailored for patient 100 and that can be the basis for future feedback, for example, future alerts and activation of environment control systems. Auto regression and moving average processes may be invoked to model observed data and generate predictive models.

Data analysis module 440 outputs respiratory health data on user interface 310, and may also transmit respiratory health data via communication network 120 for output on clinician computer 130 or family computer 140. Output respiratory health data may include present health data, such as current wheeze rate, crackle rate, pulse rate, respiratory rate, inspiratory duration, expiratory duration, SpO2 level, airborne particulate levels, ambient temperature or relative humidity and/or patient-friendly respiratory health score. Output respiratory health data may also include health trend data, such as up or down arrows for components of present health data.

Data analysis module 440 also generates and outputs respiratory health alerts and environment control messages in response to respiratory health data. Data analysis module 440 generates respiratory health alerts and/or environment control messages in response to respiratory health data that exceeds or falls below configured alarm and/or control thresholds. Alarm/control thresholds may be established for comparison with present health data or health trend data for individual scientific parameters (e.g. current or trend for wheeze rate, crackle rate, pulse rate, respiratory rate, inspiratory duration, expiratory duration, SpO2 level, airborne particulate levels, ambient temperature and/or relative humidity), groups of scientific parameters or the patient-friendly respiratory health score. For example, if a patient-friendly respiratory health score falls to one (i.e. on a scale of one to five with one being lowest), an alarm may be triggered that causes data analysis module 440 to output an audible and/or visual respiratory health alert to patient 100 via user interface 310 and also transmit a respiratory health alert for output on clinician computer 130 and/or family computer 140. As another example, where environment control system 150 is a ventilation system, if airborne particle density rises above a configured level a control may be triggered that causes data analysis module 440 to transmit an environment control message to environment control system 150 instructing the system to activate. Respiratory health alerts may indicate the reason for the alert (e.g. “patient X respiratory health score too low”) and may also make a specific recommendation (e.g “stop running”, “leave this environment”, “take medication”, “go to emergency room”). Alarm/control thresholds may be configured on handset 110 through input by patient 100 on user interface 310 or may be configured remotely by a clinician. In other embodiments, alarm/control thresholds may be automatically configured by data analysis module 440 through application of patient background data to a predictive model operative on data analysis module 440. In response to receiving a respiratory health alert, a clinician may upload present health data and health trend data to clinician computer 130 for detailed diagnosis.

In some embodiments, in addition to or in lieu of the above respiratory health alarms/controls, respiratory health alerts and environment control messages may be generated through application of respiratory health data to a predictive model operative on data analysis module 440 that continually calculates a probability of an asthma attack using patient background data, present health data and health trend data. If the calculated probability exceeds a probability threshold, a respiratory health alert or environment control message may be generated.

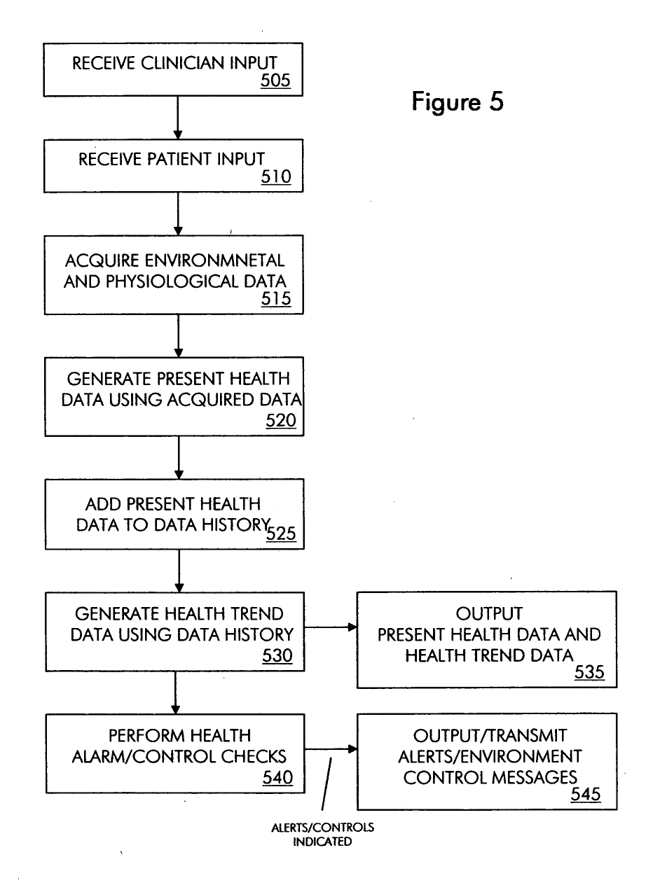

FIG. 5 shows a method for respiratory health self-monitoring in some embodiments of the invention. Clinician input is uploaded to handset 110 (505) and patient input is input to handset 110 (510). Clinician input and patient input include, for example, patient background data, alarm/control thresholds and any supplemental physiological data (e.g. lung performance data obtained using a peak flow meter). Handset 110 then acquires via BAN 210 environmental and physiological data from monitors 220, 230, 240, 250 at regular intervals (515) and converts the acquired environmental and physiological data to the extent necessary. Handset 110 generates present health data using the acquired environmental and physiological data (520) and adds the present health data to a data history (525). Present health data includes, for example, scientific parameter values such as current wheeze rate, crackle rate, pulse rate, respiratory rate, inspiratory duration, expiratory duration, SpO2 level, airborne particulate levels, ambient temperature and relative humidity; and a patient-friendly respiratory health score. Handset 110 generates health trend data using the data history (530). Health trend data includes, for example, up or down arrows associated with scientific parameter values. Handset 110 outputs present health data and health trend data (535). Handset 110 performs respiratory health alarm/control checks (540) and outputs/transmits respiratory health alerts and environment control messages if indicated (545).

It will be appreciated by those of ordinary skill in the art that the invention can be embodied in other specific forms without departing from the spirit or essential character hereof. For example, in some embodiments, the handset may be replaced by a mobile electronic device that is not handheld, such as a notebook computer. Moreover, although the invention has been described in connection with management of asthma, the invention is readily applicable to other diseases, such as Rhinitis. The present description is therefore considered in all respects to be illustrative and not restrictive. The scope of the invention is indicated by the appended claims, and all changes that come with in the meaning and range of equivalents thereof are intended to be embraced therein.

What is claimed is:

1. A method for respiratory health self-monitoring, comprising the steps of:
receiving physiological data collected from a patient;
receiving environmental data; and
generating respiratory health data for the patient based at least in part on the physiological data and the environmental data.

2. The method of claim 1, wherein the physiological data and the environmental data comprise data received on a mobile electronic device at regular intervals.

3. The method of claim 2, wherein the physiological data further comprise data received on a mobile electronic device episodically.

4. The method of claim 1, wherein the respiratory health data are further generated based at least in part on statically configured patient background data.

5. The method of claim 4, wherein patient background data comprise at least one of the behavior pattern data, co-morbidity data, medication data, age data, height data, weight data, gender data, race data or genetic background data.

6. The method of claim 1, wherein the respiratory health data comprise present health data generated using current physiological data and environmental data.

7. The method of claim 1, wherein the respiratory health data comprise health trend data generated using historical physiological data and environmental data.

8. The method of claim 1, wherein the respiratory health data comprise health cross-correlation data generated using historical physiological data and environmental data.

9. The method of claim 1, further comprising the step of outputting a respiratory health alert in response to the respiratory health data.

10. The method of claim 1, further comprising the step of controlling an environment control system in response to the respiratory health data.

11. The method of claim 1, further comprising the step of generating a predictive model for the patient in response to the respiratory health data.

12. The method of claim 1 wherein the physiological data comprise at least one of lung sound data, blood oxygen saturation (SpO2) data or pulse rate data.

13. The method of claim 1, wherein the environmental data comprise at least one of airborne particulate data, temperature data or relative humidity data.

14. The method of claim 1, wherein the environmental data comprise at least one of airborne particulate presence, type or density data.

15. A handset, comprising:
at least one network interface; and
a processor communicatively coupled with the network interface, wherein the network interface is adapted to receive at regular intervals via a wireless link physiological data from at least one physiological monitor and environmental data from at least one environmental monitor and the processor is adapted to generate respiratory health data for a patient operatively coupled to the at least one physiological monitor based at least in part on the physiological data and the environmental data.

16. A body area network (BAN), comprising:
at least one physiological monitor operatively coupled to a patient;
at least one environmental monitor; and
a handset communicatively coupled with the physiological monitor and the environmental monitor, wherein the handset generates respiratory health data for the patient based at least in part on physiological data acquired by the handset at regular intervals from the physiological monitor and the environmental monitor.

17. The BAN of claim 16, wherein the respiratory health data are further generated based at least in part on patient background data statically configured on the handset.

18. The BAN of claim 16, wherein the handset outputs the respiratory health data on a user interface of the handset.

19. The BAN of claim 16, wherein the handset outputs a respiratory health alert in response to the respiratory health data.

20. The BAN of claim 16, wherein the handset transmits an environment control message from the handset in response to the respiratory health data.
